(19) United States
(12) Patent Application Publication
(10) Pub. No.: US 2006/0172329 A1
(43) Pub. Date: Aug. 3, 2006

(54) DNA markers for cattle growth

(75) Inventors: Jeremy F. Taylor, Columbia, MO (US); Robert D. Schnabel, Columbia, MO (US)

Correspondence Address: FULBRIGHT & JAWORSKI L.L.P., 600 CONGRESS AVE., SUITE 2400, AUSTIN, TX, 78701, US

(73) Assignee: The University of Missouri System

(21) Appl. No.: 11/329,973

(22) Filed: Jan. 11, 2006

Related U.S. Application Data
(60) Provisional application No. 60/643,683, filed on Jan. 13, 2005.

(51) Int. Cl. C12Q 1/68; C12P 19/34

(52) U.S. Cl. 435/6; 435/91.2

(57) ABSTRACT

The invention provides methods for identifying a genetic polymorphism associated with increasing weaning weight in progeny of female beef cattle comprising the polymorphism. Genetic marker-assisted selection methods provided by the invention allow avoidance of potentially costly phenotypic testing and inaccuracies associated with traditional breeding schemes and improvement of beef cattle herds.

This application claims benefit of and priority to U.S. Provisional Patent Application No. 60/643,683, filed Jan. 13, 2005, which is herein incorporated by reference in its entirety.

### BACKGROUND OF THE INVENTION

1. Field of the Invention

The present invention relates generally to the field of mammalian genetics. More particularly, it concerns genetic markers for the selection of cattle having a genetic predisposition for progeny with superior growth traits.

2. Description of Related Art

It has proven to be extremely difficult to identify the causal mutations underlying livestock quantitative trait loci (QTLs) and this has severely handicapped the application of marker-assisted selection (MAS) in commercial livestock species. The availability of a whole genome sequence has been expected to assist in the identification of candidate genes within a critical region harboring a QTL and also in the design of polymerase chain reaction primers to screen for diversity within coding and non-coding regulatory regions of targeted candidate genes. However, this has not overcome the key problem for quantitative trait nucleotide (QTN) identification; the recognition of the important regulatory regions and the identification of causal mutations within these regions.

The Human Genome Project (HGP) began in 1990 with the expectation that the sequence of the human genome would reveal the genetic mechanisms underlying human variation, particularly disease, and lead to new therapeutics that could be individually customized according to a patient's genotype. The advent of the HGP unleashed a similar biotechnological fervor in animal agriculture.

One area of success has been the identification of QTLs associated with milk quality and quantity. A non-conservative lysine to alanine substitution (K232A) in the bovine and acylCoA:diacylglycerol acyltransferase (DGAT1) gene has been shown to be the causative mutation affecting variation in milk yield and composition traits of Holstein cows (Grisart et al., 2002, 2004; U.S. Patent Appl. Pub. No. 20040076977). The alanine allele produces an increase in overall milk yield and protein, but also decreases milk fat. Although the alanine allele is under positive selection in the U.S. Holstein population, in which overall milk yield has been primarily selected for, the lysine allele has been selected for in New Zealand dairy cattle populations, where increased milk fat is of primary economic importance (Spelman et al., 2002).

While the foregoing has been beneficial for the improvement of dairy cattle, techniques for the improvement of beef cattle have been largely lacking. The list of traits important to beef cattle production differs from dairy cattle and is extensive. However, little genetic improvement for meat quality or the efficiency of production has occurred in beef cattle populations in the last 100 years despite development of Selection Index theory over 60 years ago. This is due at least in part to the little information available on which to make selection decisions to improve these traits. It is extremely difficult and costly to obtain carcass information in commercial packing plants and to retain the identity of individual animals. Accordingly, very few beef breed associations have been able to develop expected progeny differences (EPDs), a statistical estimate of the average additive genetic value of a gamete produced by an individual, and considerable efforts have been expended to develop live animal ultrasound techniques to provide indirect measures of carcass traits. Little to no information is therefore available upon which to make breeding decisions to improve the net efficiency of growth. Due to the importance of these traits and their cost and difficulty of measurement, there is a great need for development of indirect measures for selection of beneficial traits in beef cattle such as DNA diagnostics. Such techniques could greatly increase the productivity of breeding programs and eliminate the need for costly or ineffective phenotypic selections.

### SUMMARY OF THE INVENTION

In one aspect, the invention provides a method of obtaining a female head of beef cattle comprising a genetic predisposition for yielding progeny with increased weaning weight, the method comprising the steps of: (a) genotyping at least a first female head of beef cattle for a genetic polymorphism in DGAT1 associated with increased weaning weight in progeny of female beef cattle comprising the polymorphism; and (b) selecting a female head of beef cattle having the polymorphism. In particular embodiments of the invention, the genetic polymorphism may be further defined as a lysine to alanine substitution (K232A) in the bovine DGAT1 gene. Genotyping the first female parent head of beef cattle for the presence of the genetic polymorphism in DGAT1 may comprise, in addition to direct testing of the female parent, testing of one or both of the parents of the female parent to determine the genotype of the first female parent.

Such a polymorphism may be detected by any method, both at the nucleic acid and protein level. One convenient method for detection comprises use of the polymerase chain reaction. This and other techniques are well known to those of skill in the art as described herein below. Genetic material assayed may comprise, for example, genomic DNA or RNA. This can be obtained from cattle post-birth, or may be obtained from fetal animals, including from embryos in vitro. The selecting may comprise embryo transfer of the embryo, such that the first head of beef cattle is grown from the embryo. The methods of the invention may be used in connection with any type of beef cattle, including Bos indicus and Bos taurus cattle.

In yet another aspect, the invention provides a method of breeding cattle to increase the probability of obtaining a progeny head of beef cattle having increased weaning weight, the method comprising the steps of: (a) selecting a first female parent head of beef cattle for the presence of a genetic polymorphism in DGAT1, wherein the polymorphism is associated with increased weaning weight in progeny of female beef cattle comprising the polymorphism; and (b) breeding the first parent head of beef cattle with a male parent head of beef cattle to obtain at least a first progeny head of beef cattle comprising increased weaning weight relative to a progeny of a female head of beef cattle lacking the polymorphism. The method may further comprise selecting the second parent head of beef cattle based on the genetic polymorphism in DGAT1. Selecting the first female parent head of beef cattle for the presence of the genetic polymorphism in DGAT1 may comprise direct testing of the female parent, as well as one or both of the parents of the female parent.

In one aspect of the invention, the foregoing techniques may be “reversed” and DGAT1 genotype selection is used to obtain an allele that decreases weaning weight of calves through selection of parents with the appropriate DGAT1 genotypes. Such a selection may be used, for example, to provide other benefits, including more efficient energy utilization by female animals and hardiness of animals. The invention therefore encompasses the foregoing methods wherein the lysine allele at amino acid 232 of DGAT1 is selected for. In one aspect of the invention, a method is therefore provided comprising (a) genotyping at least a first female head of beef cattle for a genetic polymorphism in DGAT1 associated with decreased weaning weight in progeny of female beef cattle comprising the polymorphism; and (b) selecting a female head of beef cattle having the polymorphism. In particular embodiments of the invention, the genetic polymorphism may be further defined as a K232 allele. The invention therefore also provides a method comprising the steps of: (a) selecting a first female parent head of beef cattle for the presence of a genetic polymorphism in DGAT1 associated with decreased weaning weight in progeny of female beef cattle comprising the polymorphism; and (b) breeding the first parent head of beef cattle with a male parent head of beef cattle to obtain at least a first progeny head of beef cattle comprising decreased weaning weight relative to a progeny of a female head of beef cattle lacking the polymorphism.

In a method of the invention, one or both of the first parent head of beef cattle and the second parent head of beef cattle may be any beef cattle type, for example a Bos indicus or Bos taurus head of beef cattle. The method may still further be defined as comprising crossing a progeny head of beef cattle with a third head of beef cattle to produce a second generation progeny head of beef cattle. The third head of beef cattle may be a parent of the progeny head of beef cattle or may be unrelated to the progeny head of beef cattle. In certain embodiments of the invention, the aforementioned steps are repeated from about 2 to about 10 times, wherein the first parent head of beef cattle is selected from a progeny head of beef cattle resulting from a previous repetition of step (a) and step (b) and wherein the second parent head of beef cattle is from a selected cattle breed into which one wishes to introduce increased weaning weight. This technique will therefore allow, for example, the introduction of the beneficial characteristic into a genetic background otherwise lacking the trait but possessing other desirable traits.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Shows interval analyses (Haldane cM) for Angus milk EPD for cattle chromosome 14 which contains the DGAT1 gene.

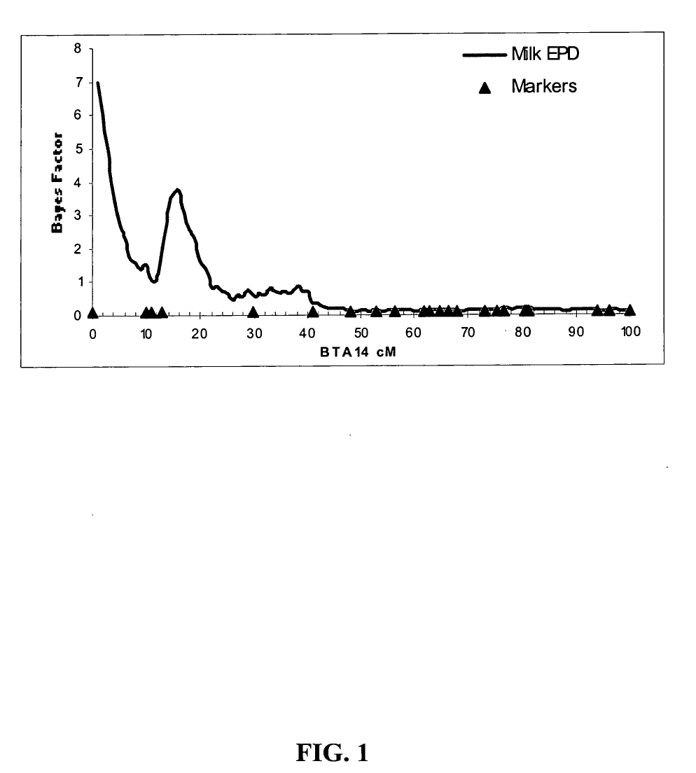

### DESCRIPTION OF ILLUSTRATIVE EMBODIMENTS

The invention provides, in one aspect, methods and compositions for the improvement of beef cattle with respect to the weaning weight of progeny obtained from the beef cattle. It was surprisingly found that the non-conservative lysine to alanine substitution (K232A) in the bovine acylCoA:diacylglycerol acyltransferase (DGAT1) gene responsible for milk yield and composition in Holstein dairy cows causes variation in the weaning weight of beef cattle. The milk EPD of bulls that were homozygous for an Alanine allele in DGAT1 was found to average 6.31 lb higher for weaning weight than those homozygous for a Lysine allele at the same locus. Therefore, daughters from bulls that were homozygous for the Alanine allele weaned calves, on average 6.31 lb heavier than daughters from bulls that were homozygous for the Lysine allele after accounting for the genes that directly affect an animal's growth.

The techniques of the invention are significant in that they allow improvement of beef cattle for a previously unidentified beef cattle trait without the need for costly or unreliable phenotypic assays and manual breeding selections. With the increasing costs associated with animal breeding and artificial insemination, each head of cattle produced represents a substantial investment of time and money. Traditional methods of breeding cattle have included standard breeding techniques in which sire progenies are studied. However, such techniques can lack accuracy due to environmental variance or scoring error. Further, complex gene action and interactions among genes can complicate breeding. Phenotypic selection often does not efficiently take into account such genetic variability. Selection based upon DNA tests is therefore significant in that it allows improvement of beef cattle for progeny weaning weight without the cost and lack of reliability of conventional assays or selections.

The use of genetic assays to identify the polymorphisms identified herein as associated with increased weaning weight will find use in breeding or selecting of beef cattle produced for slaughter, e.g., for production of meat products. Thus, one embodiment of the invention comprises a breeding program directed at enhancement of growth characteristics in beef cattle breeds adapted for meat production, as opposed to cattle specifically suited or used for production of dairy products. Such techniques have to date been largely lacking for beef cattle.

While the utilization of B. taurus×B. indicus crosses has resulted in the detection of numerous QTL affecting growth and carcass composition, it does not seem to have assisted in the identification of the causal mutations underlying these QTL effects. This may be due to a combination of factors including: 1) the lack of whole genome sequence for cattle, 2) limited experience in the identification of regulatory mutations associated with transcription, alternative splicing, mRNA stability and localization, or the efficiency of translation, and 3) the occurrence of SNPs with fixed allelic differences between B. taurus and B. indicus as frequently as every 1700 bp of coding sequence leading to enormous difficulty in eliminating candidates for causal mutations since genome scans can resolve the location of a QTL only to a chromosome interval of 5-20 Mb which contains from 60-240 genes (Heaton et al., 2001; White et al., 2003). The task of elucidating causal SNP(s) is therefore extremely difficult and has hampered implementation of marker-assisted selection outside of the population in which a QTL is initially detected since the QTL allele frequencies and marker-QTL allele phase relationships are unknown in the commercial populations in which improvement is desired. The availability of genetic assays for beneficial beef cattle traits therefore represents a significant advance.

### I. Genetic Assays and Selections

Genetic assay-assisted selections for animal breeding are important in that they allow selections to be made without the need for raising and phenotypic testing of progeny. In particular, such tests allow selections to occur among related individuals that do not necessarily exhibit the trait in question and that can be used in introgression strategies to select both for the trait to be introgressed and against undesirable background traits (Hillel et al., 1990). However, it is has been difficult to identify genetic assays for loci yielding highly heritable traits of large effect, particularly as many such traits may not be segregating and already be fixed with near optimal alleles in commercial lines. The invention overcomes this difficulty by providing such assays for alleles that are segregating in beef cattle populations.

In accordance with the invention any assay which sorts and identifies animals based upon DGAT1 allelic differences may be used and is specifically included within the scope of this invention. One of skill in the art will recognize that, having identified a causal polymorphism for a particular associated trait, there are an essentially infinite number of ways to genotype animals for this polymorphism. These tests may be made at the nucleic acid and protein level. The design of such alternative tests merely represents a variation of the techniques provided herein and is thus within the scope of this invention as fully described herein. Illustrative procedures are described herein below.

Non-limiting examples of methods for identifying the presence or absence of a polymorphism include single-strand conformation polymorphism (SSCP) analysis, RFLP analysis, heteroduplex analysis, denaturing gradient gel electrophoresis, temperature gradient electrophoresis, ligase chain reaction and direct sequencing of the gene. Techniques employing PCR™ detection are advantageous in that detection is more rapid, less labor intensive and requires smaller sample sizes. Primers that may be used in this regard are disclosed in U.S. Patent Appl. Pub. No. 20040076977, the disclosure of which is incorporated herein by reference in its entirety. A PCR™ amplified portion of the DGAT1 gene can be screened for a polymorphism, for example, with direct sequencing of the amplified region, by detection of restriction fragment length polymorphisms produced by contacting the amplified fragment with a restriction endonuclease having a cut site altered by the polymorphism, by allele specific PCR™ in which the lysine and alanine alleles are individually amplified by specific oligonucleotide primers as well as by SSCP analysis of the amplified region. These techniques may also be carried out directly on genomic nucleic acids without the need for PCR™ amplification, although in some applications this may require more labor.

Once an assay format has been selected, selections may be unambiguously made based on genotypes assayed at any time after a nucleic acid or protein sample can be collected from an individual, such as an infant animal, or even earlier in the case of testing of embryos in vitro, or testing of fetal offspring. Any source of genetic material (including, for example, DNA and RNA) or a product encoded thereby may be analyzed for scoring of genotype. In one embodiment of the invention, nucleic acids are screened that have been isolated from the hair roots, blood or semen of the bovine analyzed. Generally, peripheral white blood cells are conveniently used as the source, and the genetic material is DNA. A sufficient amount of cells are obtained to provide a sufficient amount of DNA for analysis, although only a minimal sample size will be needed where scoring is by amplification of nucleic acids. The DNA can be isolated from the blood cells by standard nucleic acid isolation techniques known to those skilled in the art.

In genetic assay-assisted breeding, eggs may be collected from selected females and in vitro fertilized using semen from selected males and implanted into other females for birth. Assays may be advantageously used with both male and female cattle. Using in vitro fertilization, genetic assays may be conducted on developing embryos at the 4-8 cell stage, for example, using PCR™, and selections made accordingly. Embryos can thus be selected that are homozygous for the desired marker prior to embryo transfer.

Use of genotype-assisted selection provides more efficient and accurate results than traditional methods. This also allows rapid introduction into or elimination from a particular genetic background of the specific trait or traits associated with the identified genetic marker. In the instant case, screening for DGAT1 alleles conferring increased or decreased weaning weight may be used to allow the efficient culling of females that will wean calves at lower weights, and the selection of bulls and cows that will produce daughters which will wean calves of higher weaning weight, as desired.

Genetic assays can be used to obtain information about the genes that influence an important trait, thus facilitating breeding efforts. Factors considered in developing markers for a particular trait include: how many genes influence a trait, where the genes are located on the chromosomes (e.g., near which genetic markers), how much each locus affects the trait, whether the number of copies has an effect (gene dosage), pleiotropy, environmental sensitivity and epistatis.

A genetic map represents the relative order of genetic markers, and their relative distances from one another, along each chromosome of an organism. During sexual reproduction in higher organisms, the two copies of each chromosome pair align themselves closely with one another. Genetic markers that lie close to one another on the chromosome are seldom recombined, and thus are usually found together in the same progeny individuals. Markers that lie close together show a small percent recombination, and are said to be linked. Markers linked to loci having phenotypic effects are particularly important in that they may be used for selection of individuals having the desired trait.

The identity of a given allele can therefore be determined by identifying nearby genetic markers that are usually co-transmitted with the gene from parent to progeny. This principle applies both to genes with large effects on phenotype (simply inherited traits) and genes with small effects on phenotype. As such, by identifying a marker linked to a particular trait, this will allow direct selection for the linked polymorphism without the need for detecting that particular polymorphism due to genetic linkage between the traits. Those of skill in the art will therefore understand that when genetic assays for DGAT1 are mentioned herein this specifically encompasses detection of genetically linked polymorphisms that are informative for the DGAT1 allele. Such polymorphisms have predictive power relative to the trait to the extent that they also are linked to the contributing locus for the trait. Such markers thus also have predictive potential for the trait of interest.

Most natural populations of animals are genetically quite different from the classical linkage mapping populations. While linkage mapping populations are commonly derived from two-generation crosses between two parents, many natural populations are derived from multi-generation matings between an assortment of different parents, resulting in a massive reshuffling of genes. Individuals in such populations carry a complex mosaic of genes, derived from a number of different founders of the population. Gene frequencies in the population as a whole may be modified by a natural or artificial selection, or by genetic drift (e.g., chance) in small populations. Given such a complex population with superior average expression of a trait, a breeder might wish to (1) maintain or improve the expression of the trait of interest, while maintaining desirable levels of other traits; and (2) maintain sufficient genetic diversity that rare desirable alleles influencing the trait(s) of interest are not lost before their frequency can be increased by selection.

Genetic assays may find particular utility in maintaining sufficient genetic diversity in a population while maintaining favorable alleles. For example, one might select a fraction of the population based on favorable phenotype (perhaps for several traits—one might readily employ index selection), then apply genetic assays as described herein to this fraction and keep a subset which represent much of the allelic diversity within the population. Strategies for extracting a maximum of desirable phenotypic variation from complex populations remain an important area of breeding strategy. An integrated approach, merging classical phenotypic selection with a genetic marker-based analysis, may aid in extracting valuable genes from heterogeneous populations.

The techniques of the present invention may potentially be used with any bovine, including Bos taurus and Bos indicus cattle. In particular embodiments of the invention, the techniques described herein are specifically applied for selection of beef cattle, as the genetic assays described herein will find utility in maximizing production of animal products, such as meat. As used herein, the term “beef cattle” refers to cattle grown or bred for production of meat or other non-dairy animal products. Therefore, a “head of beef cattle” refers to at least a first bovine animal grown or bred for production of meat or other non-dairy animal products. Examples of breeds of cattle that may be used with the invention include, but are not limited to, Africander, Albères, Alentejana, American, American White Park, Amerifax, Amrit Mahal, Anatolian Black, Andalusian Black, Andalusian Grey, Angeln, Angus, Ankole, Ankole-Watusi, Argentine Criollo, Asturian Mountain, Asturian Valley, Australian Braford, Australian Lowline, Ba-Bg, Bachaur, Baladi, Barka, Barzona, Bazadais, Beefalo, Beefmaker, Beefmaster, Belarus, Red, Belgian Blue, Belgian Red, Belmont Adaptaur, Belmont Red, Belted Galloway, Bengali, Berrendas, Bh-Bz, Bhagnari, Blanco Orejinegro, Blonde d'Aquitaine, Bonsmara, Boran, Braford, Brahman, Brahmousin, Brangus, Braunvieh, British White, Busa, Cachena, Canary Island, Canchim, Carinthian Blond, Caucasian, Channi, Charbray, Charolais, Chianina, Cholistani, Corriente, Costeño con Cuernos, Dajal, Damietta, Dangi, Deoni, Devon, Dexter, Dhanni, Dølafe, Droughtmaster, Dulong, East Anatolian Red, Enderby Island, English Longhorn, Evolene, Fighting Bull, Florida Cracker/Pineywoods, Galician Blond, Galloway, Gaolao, Gascon, Gelbray, Gelbvieh, German Angus, German Red Pied, Gir, Glan, Greek Shorthorn, Guzerat, Hallikar, Hariana, Hays Converter, Hereford, Herens, Highland, Hinterwald, Holando-Argentino, Horro, Hungarian Grey, Indo-Brazilian, Irish Moiled, Israeli Red, Jamaica Black, Jamaica Red, Jaulan, Kangayam, Kankrej, Kazakh, Kenwariya, Kerry, Kherigarh, Khillari, Krishna Valley, Kurdi, Kuri, Limousin, Lincoln Red, Lohani, Luing, Maine Anjou, Malvi, Mandalong, Marchigiana, Masai, Mashona, Mewati, Mirandesa, Mongolian, Morucha, Murboden, Murray Grey, Nagori, N'dama, Nelore, Nguni, Nimari, Ongole, Orma Boran, Oropa, Parthenais, Philippine Native, Polish Red, Polled Hereford, Ponwar, Piedmontese, Pinzgauer, Qinchuan, Ratien Gray, Rath, Rathi, Red Angus, Red Brangus, Red Poll, Retinta, Rojhan, Romagnola, Romosinuano, RX3, Sa-Sg, Sahiwal, Salers, Salorn, Sanhe, Santa Cruz, Santa Gertrudis, San Martinero, Sarabi, Senepol, Sh-Sz, Sharabi, Shorthorn, Simbrah, Simmental, Siri, Slovenian Cika, South Devon, Sussex, Swedish Red Polled, Tarentaise, Telemark, Texas Longhorn, Texon, Tharparkar, Tswana, Tuli, Ukrainian Beef, Ukrainian Grey, Ukrainian Whitehead, Umblachery, Ural Black Pied, Vestland Red Polled, Vosges, Wagyu, Welsh Black, White Caceres, White Park, Xinjiang Brown and Yanbian cattle breeds, as well as animals bred therefrom and related thereto.

### II. Nucleic Acid Detection

Techniques for nucleic acid detection may find use in certain embodiments of the invention. For example, such techniques may find use in scoring individuals for genotypes or in the development of novel markers linked to the major effect locus identified herein.

1. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, nucleotide sequences may be used in accordance with the invention for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, lower stringency conditions may be preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M NaCl, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences with the present invention in combination with an appropriate means, such as a label, for determining hybridization. For example, such techniques may be used for scoring of RFLP marker genotype. A wide variety of appropriate indicator means are known in the art, including fluorescent, radioactive, enzymatic or other ligands, such as avidin/biotin, which are capable of being detected. In certain embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that probes or primers will be useful as reagents in solution hybridization, as in PCR™, for detection of nucleic acids, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

2. Amplification of Nucleic Acids

Nucleic acids used as a template for amplification may be isolated from cells, tissues or other samples according to standard methodologies (Sambrook et al., 1989). Such embodiments may find particular use with the invention, for example, in the detection of repeat length polymorphisms, such as microsatellite markers. In certain embodiments of the invention, amplification analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer”, as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles”, are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Affymax technology; Bellus, 1994). Typically, scoring of polymorphisms as fragment length variants will be done based on the size of the resulting amplification product.

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to obtain cDNA, which in turn may be scored for polymorphisms. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR and oligonucleotide ligase assay (OLA), disclosed in U.S. Pat. No. 5,912,148, also may be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, also may be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site also may be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., 1990; PCT Application WO 88/10315, incorporated herein by reference in their entirety). European Application No. 329 822 disclose a nucleic acid amplification process involving cyclically synthesizing single-stranded RNA (ssRNA), single-stranded DNA (ssDNA), and double-stranded DNA (dsDNA), which may be used in accordance with the present invention.

PCT Application WO 89/06700 (incorporated herein by reference in its entirety) discloses a nucleic acid sequence amplification scheme based on the hybridization of a promoter region/primer sequence to a target ssDNA followed by transcription of many RNA copies of the sequence. This scheme is not cyclic, i.e., new templates are not produced from the resultant RNA transcripts. Other amplification methods include “race” and “one-sided PCR™” (Frohman, 1990; Ohara et al., 1989).

3. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids also may be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 1989). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Other Assays

Other methods for genetic screening may be used within the scope of the present invention, for example, to detect polymorphisms in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substitution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

5. Kits

All the essential materials and/or reagents required for screening cattle for genetic marker genotype in accordance with the invention may be assembled together in a kit. This generally will comprise a probe or primers designed to hybridize specifically to individual nucleic acids of interest in the practice of the present invention, for example, primer sequences such as those for amplifying DGAT1. Also included may be enzymes suitable for amplifying nucleic acids, including various polymerases (reverse transcriptase, Taq, etc.), deoxynucleotides and buffers to provide the necessary reaction mixture for amplification. Such kits also may include enzymes and other reagents suitable for detection of specific nucleic acids or amplification products. Such kits generally will comprise, in suitable means, distinct containers for each individual reagent or enzyme as well as for each probe or primer pair.

### III. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Resource Populations, Phenotypes and EPDs

A Repository was created for DNA samples derived from at least 1 straw or ampule of semen (˜360 μg DNA/sire on average) on 1,555 related Angus bulls which span 14 generations with the oldest bull born in 1956. This population represents the major sire lineages within the Angus breed and was designated the Missouri Angus Pedigree (MAP) population. Two sons of Band 234 of Ideal 3163; Tehama Bando 155 (#9891499) and Q A S Traveler 23-4 (#9250717) were popular Angus sires and have 21 and 29 sons, respectively, in the Repository. N Bar Emulation EXT (#10776479) had the largest number of sons (N=69) in the Repository. All sires except family probands had DNA available on their sires and 77.9% also had DNA represented on their maternal grandsire. All pedigree data, EPDs and reliabilities for 20 traits were obtained from the American Angus Association for these bulls.

Additionally, a collection was obtained from the Circle A Ranch of Iberia, Mo. comprising DNA samples (˜110 μg/steer) and a database of pedigree and phenotypic records on 5,485 pedigreed Angus steer progeny produced in the Circle A Angus Sire Alliance by mating to registered Angus sires represented in the MAP. Steer phenotypic data available for the population included weights [birth (N=4,572); weaning (4,562)], live animal ultrasound measures [weight (4,257); fat thickness (4,264); ribeye area (4,267); intramuscular fat % (4,267)], carcass measures [ribeye area (4,551); USDA marbling score (4,564); yield grade (4,526); fat thickness (4,549); hot carcass weight (4,592)] and all weigh dates and contemporary group identifiers. Growth and individual animal feed intake data were available for 561 steers with DNA samples and carcass data (N=561 ultrasound; N=341 carcass). Within each feeding contemporary group, a residual feed intake (RFI) was calculated for steers using a partial regression model in which average daily feed intake was regressed on average daily gain and metabolic mid-weight [(weight at mid-feeding period)^{0.75}] (Herd et al., 2003).

### Example 2

### QTL Analysis for Growth-Associated Loci

BTA2 and BTA14 were examined as possible locations for identification of new growth-associated QTLs. There was also an interest in scoring the SNP mutations in acylCoA:diacylglycerol acyltransferase (DGAT1) (Grisart et al., 2002; 2004) to determine whether DGAT1 polymorphisms were segregating in Angus cattle, and if so, to test for possible phenotypic effects in beef cattle. Consequently, DGAT1 was specifically examined as a candidate QTL for phenotypic variation in Angus cattle.

An examination was therefore initiated on the impact of calf weaning weight by QTL variations in DGAT1 in beef cattle. Microsatellites were first chosen from the published genetic maps that possess a large number of alleles that could be efficiently scored (Barendse et al., 1997, www.cgd.csiro.au/cgd.html; Kappes et al., 1997, www.marc.usda.gov/genome/genome.html). The forward PCR™ primer for each marker was synthesized with one of 4 fluorescent dye labels. Multiplexed PCR™s were developed based on the allele size ranges, fluorescent label and the empirically determined ability of each marker to co-amplify. Multiplex-PCR™ was performed using 5 μl reactions on an ABI 9700 thermocycler (Applied Biosystems Inc., Foster City, Calif.) as described by Schnabel et al., (2003). PCR™ products were separated on either an ABI 3100 or ABI 3700 Automated Sequencer and sized relative to the GS500 LIZ internal size standard (Applied Biosystems). Fluorescent signals from the dye-labeled microsatellites were detected using GENESCAN v3.1 (Applied Biosystems) and genotypes were assigned using Genotyper v3.7 (Applied Biosystems).

DNA was extracted from all 1,555 MAP sires and from the 5,485 Circle A Angus steers and 5×384-well master plates were created for 1,361 of the MAP sires and 559 steers with feed intake and RFI data. The markers were not prescreened for informativeness due to the multi-generation structure of the MAP and thus there were concerns that many microsatellites might not be informative in this purebred pedigree. Consequently, it was chosen to score markers at a high resolution (4 cM) to estimate the proportion of informative markers and to be assured of producing maps at an average resolution of no less than 10 cM (40% of markers informative) with no large inter-marker intervals. The microsatellite-based scan of BTA2 and BTA14 in these 1,920 males with 56 multiplexed microsatellite markers and mutations in TG5 (Barendse et al., 2001) and DGAT1 (Grisart et al., 2002; 2004) producing 113,637 after misinheritances were corrected and missing genotypes were inferred using GENOPROB (Thallman et al. 2001a,b). TG5 and DGAT1 were genotyped as PCR™ RFLPs and scored on agarose gels: 1.5% for DGAT1 and 3% for TG5 (50% standard agarose and 50% high resolution NuSieve 3:1 agarose (Cambrex Bioscience, Rockland, Me.)).

The overall rate of missing genotypes due to failed PCR™ or failed injections (on an ABI 3700) was 4.6% and no attempt was made to rerun missing genotypes. On average, 5.8 loci were amplified in each reaction, however, 2 multiplexes were run with only 2 markers each. Complete pedigree information linking all genotyped animals was assembled to exploit the relationships among Angus. Genotype and grand-parental origin probabilities were estimated for each of the genotyped animals using genotype, map and pedigree information. Individual genotypes with low probability (pGmx<0.98) estimated by GENOPROB (Thallman et al., 2001a,b) were excluded from further analysis. Only 2 markers could not be incorporated in the genetic maps due to a lack of informative meioses (IM) and 36 (64%) produced at least 1,000 IM. Across all 58 loci, the average number of IM exceeded 1,180. In 1,737 Angus MAP sires and Circle A steers, the frequency of the lysine DGAT1 allele was 14.8%, with 1.6% of animals homozygous for this allele.

### Example 3

### DGAT1 Polymorphisms Segregate in Beef Cattle and Contribute Significant Variation in Growth Rate of Calves From Dams With Differing QTL Genotypes

The DGAT1 K232A mutation was detected as a polymerase chain reaction-restriction fragment length polymorphism on 1.5% agarose gels in an extended pedigree of 1,361 artificial insemination Angus sires from the Missouri Angus Pedigree population described in Example 1. A total of 1,250 DGAT1 genotypes were assigned pGmx>0.98 by GENOPROB and were used in subsequent analyses. Genotyping was also carried out of a SNP within the Thyrogobulin gene and of 24 public microsatellite loci on BTA14 in this pedigree in order to perform a whole chromosome interval analysis, which allowed the localization of genes influencing variation in quantitative traits (QTLs) to a specific position on a chromosome. Table 1 contains the identities of the markers and their position within the genetic map of bovine chromosome 14 that were produced in this Angus mapping population.

TABLE 1
Marker identity, number of informative meioses and genetic map
of BTA14 for 24 microsatellite and 2 SNP loci in a pedigree
in which 1,920 Angus AI sires and steers were genotyped.

| | | Kosambi | Informative |
|---|---|---|---|
| BTA14¹ | Multiplex | cM | Meioses |
| DGAT | PCR-RFLP | 0.0 | 303 |
| CSSM66 | 207 | 9.0 | 990 |
| DIK4015 | 201 | 10.1 | 1521 |
| BMS1747 | 203 | 10.1 | 1442 |
| DIK4438 | 202 | 10.1 | 439 |
| TG | PCR-RFLP | 12.0 | 625 |
| RM180 | 204 | 26.9 | 602 |
| RM011 | 206 | 37.0 | 1892 |
| BMC1207 | 205 | 43.6 | 2153 |
| BL1029 | 206 | 48.3 | 2164 |
| BM1577 | 201 | 51.5 | 1078 |
| BMS108 | 205 | 56.8 | 1404 |
| BMS1304 | 200 | 57.8 | 939 |
| BMS2513 | 209 | 59.7 | 932 |
| BMS1899 | 207 | 61.0 | 1407 |
| BMS947 | 201 | 62.7 | 1296 |
| NRKM020 | 203 | 67.7 | 708 |
| NRKM005 | 209 | 69.9 | 963 |
| DIK2742 | 205 | 69.9 | 996 |
| BM4513 | 201 | 71.1 | 1440 |
| RM66 | 205 | 74.7 | 657 |
| BM2934 | 202 | 75.3 | 1575 |
| BM4305 | 204 | 75.5 | 1257 |
| BL1036 | 201 | 87.1 | 1125 |
| BM6425 | 202 | 89.3 | 1201 |
| BMS2055 | 200 | 93.0 | 1667 |
| Average | | | 1183.7 |

GENOPROB (Thallman et al., 2001a,b) and CR1-MAP (Green et al., 1990) were used to identify genotype errors, predict the missing genotypes of dams in the pedigree, construct whole chromosome linkage maps and estimate haplotypes for the DGAT1-TG5 region on BTA14. Genotype and grand-parental origin probabilities were estimated for each of the genotyped animals using genotype, map and pedigree information. Individual genotypes with low probability (pGmx<0.98) estimated by GENOPROB were excluded from further analysis.

Next, LOKI v2.4.5 (Heath, 1997) was used for multipoint QTL analysis, fitting only the QTL in the model for sire EPDs but fitting the QTL, a covariate for age and a random polygenic effect for steer phenotypes in a simultaneous analysis of BTA2 and BTA14. FIG. 2 shows the interval analysis of BTA14 for “milk EPD” in Angus. The “milk EPD” is not a measure of milk production, but rather estimates the effect of cumulative mothering ability on the weaning weight of a calf. Thus, the EPD of a bull for milk EPD represents the genetic ability of the bull to produce daughters who will wean heavier or lighter calves due to the genes for mothering ability that they inherited from their sire This figure clearly demonstrates the presence of a QTL causing variation in Angus milk EPDs located at the most centromeric marker on BTA14 which is DGAT1.

To directly estimate the effect of DGAT1 genotypes on the milk EPD of Angus sires, weighted least squares and weighted 1-way ANOVA was used with weights of 1-Accᵢ which are proportional to prediction error variances (Accᵢ is the accuracy of prediction of EPDᵢ), to estimate and test the significance of the effect of DGAT1 on milk EPD in the Angus pedigree. Results of these analyses are presented in Table 2. FIG. 2 and Table 2 demonstrate that DGAT1 causes variation in the growth rate of calves from beef cattle dams sired by bulls with differing DGAT1 genotypes. Sires homozygous for the DGAT1 alanine allele have a milk EPD that causes, on average, their daughters to wean calves 6.31 lb (or 0.58σ_{G) (P<}0.0001) heavier than sires homozygous for the lysine allele. DGAT1 explained 2% of the variance in milk EPD in the Angus population. The frequency of the lysine allele in 72 bulls born before 1980 was 26.4%, in 217 bulls born in the 1980s was 17.1%, in 312 bulls born 1990-1994 was 17.9%, in 484 bulls born 1995-1999 was 14.7% and in 165 bulls born 2000-2002 was 8.8%. Thus, it appears that the selection applied by Angus breeders on the milk EPD to increase weaning weight in the previous decade has resulted in an increase in the frequency of the alanine allele in the Angus population.

TABLE 2
Association analysis of DGAT1 with milk EPDs in Angus cattle.

| | |
|---|---|
| | Milk EPD |
| DGAT1 | (lb) |

| Genotype | LL | LA | AA | Overall |
|---|---|---|---|---|
| Mean | 10.69 | 14.00 | 17.00 | 16.02 |
| N | 24 | 347 | 879 | 1250 |
| Genotypic | 2a = | d = | | |
| Value | 6.31 | 0.16 | | |
| V_{DGAT1} | 2.35 | | | |
| 0.25*V_{A} | 117.52 | | | |
| V_{DGAT1}/ | 2.00 | | | |
| (0.25*V_{A}) | | | | |
| (%) | | | | |
| 2a/sqrt | 0.58 | | | |
| (0.25*V_{A}) | | | | |
| Genotype | F_{2.1247} = | | P < 0.0001 | |
| Test | 13.72 | | | |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Barendse W et al., 1997. A medium-density genetic linkage map of the bovine genome. Mamm Genome. 8:21-8.
Barendse W, R Bunch, M Thomas, S Armitage, S Baud and N Donaldson. 2001. The TG5 DNA marker test for marbling capacity in Australian feedlot cattle. Proc. Beef Quality CRC Marbling Symposium Oct. 9-10, 2001, Coffs Harbour pp 30-35. Available: www.beef.crc.org.au/Publications/MarblingSym/Day1/Tg5DNA.
Bellus, J. Macromol. Sci. Pure Appl. Chem., RS3241(1):1355-1376, 1994.
Boggs, D. L. and R. A. Merkel. 1984. Live Animal Carcass Evaluation and Selection Manual (2nd Ed.) Kendall/Hunt Publishing Company, Dubuque, Iowa.
Boldman, K. G., L. A. Kriese, L. D. van Vleck, and S. D. Kachman. 1993. A manual for use of MTDFREML. ARS-USDA, Clay Center, NB.
Bradley, D. G., R. T. Loftus, P. Cunningham, and D. E. Machugh. 1998. Genetics and domestic cattle origins. Evolut. Anthropol. 6:79-86.
Edens, A. and F. Talamantes. 1998. Alternative processing of growth hormone receptor transcripts. Endocrine Rev. 19:559-582.
Frohman, In: PCR Protocols: A Guide To Methods And Applications, Academic Press, N.Y., 1990.
Gebhardt F., K. S. Zanker, and B. Brandt. 1999. Modulation of epidermal growth factor gene transcription by a polymorphic dinucleotide repeat in intron 1. J. Biol. Chem. 274:13176-13180.
Gingeras et al., 1990, “Unique features of the self-sustained sequence replication (3SR) reaction in the in vitro amplification of nucleic acids,” Ann Biol Clin (Paris) 48(7):498-501.
Green P, K Falls and S Crooks. 1990. CR1-MAP documentation: Version 2.4. Washington University School, St. Louis, Mo., USA.
Grisart B, F Farnir, L Karim, N Cambisano, J J Kim, A Kvasz, M Mni, P Simon, J M Frere, W Coppieters and M Georges. 2004. Genetic and functional confirmation of the causality of the DGAT1 K232A quantitative trait nucleotide in affecting milk yield and composition. Proc Natl Acad Sci USA. 2004 101:2398-403.
Grisart B, W Coppieters, F Famir, L Karim, C Ford, P Berzi, N Cambisano, M Mni, S Reid, P Simon, R Spelman, M Georges and R Snell. 2002. Positional candidate cloning of a QTL in dairy cattle: identification of a missense mutation in the bovine DGAT1 gene with major effect on milk yield and composition. Genome Res 12:222-31.
Heath S C. 1997. Markov chain monte carlo segregation and linkage analysis for oligogenic models. Am J Hum Genet 61:748-760.
Heaton M P, W M Grosse, S M Kappes, J W Keele, C G Chitko-McKown, L V Cundiff, A Braun, D P Little and W W Laegreid. 2001. Estimation of DNA sequence diversity in bovine cytokine genes. Mamm Genome 12:32-37.
Herd R M, J A Archer and P F Arthur. 2003. Reducing the cost of beef production through genetic improvement in residual feed intake: Opportunity and challenges to application. J Anim Sci 81(E. Suppl. 1):E9-E17.
Hillel et al., 1990, Genetics 124: 783-789.
Innis et al., DNA sequencing with Thermus aquaticus DNA polymerase and direct sequencing of polymerase chain reaction-amplified DNA. Proc Natl Acad Sci U S A. 85(24):9436-9440, 1988.
Kappes S M, J W Keele, R T Stone, R A McGraw, T S Sonstegard, T P Smith, N L Lopez-Corrales and C W Beattie. 1997. A second-generation linkage map of the bovine genome. Genome Res. 7(3):235-49.
Kwoh et al., Transcription-based amplification system and detection of amplified human immunodeficiency virus type 1 with a bead-based sandwich hybridization format. Proc Natl Acad Sci USA. 86(4):1173-1177, 1989.
Lagziel A., E. Lipkin, and M. Soller. 1996. Association between SSCP haplotype at the bovine growth hormone gene and milk protein percentage. Genetics 142:945-951.
Ohara et al., 1989. One-sided polymerase chain reaction: the amplification of cDNA. Proc Natl Acad Sci USA 86:5673-5677.
Olson, M., L. Hood, C. Cantor, and D. Botstein, 1989. A common language for physical mapping of the human genome. Science 245:1434-1435.
Sambrook et al., In: Molecular Cloning: A Laboratory Manual, Vol. 1, Cold Spring Harbor Laboratory Press, Cold Spring Harbor, N.Y., Ch. 7,7.19 17.29, 1989.
Schnabel R D, J F Taylor and J N Derr. 2003. Development of a linkage map and QTL scan for growth traits in North American bison. Cytogenet Genome Res 102:59-64.
Shimajiri S., N. Nobuyuki, A. Tanimoto, Y. Murata, T. Hamada, K.-Y. Wang, Y. Sasaguri. 1999. Shortened microsatellite d(CA)21 sequence down-regulates promoter activity of matrix metaloproteinase 9 gene. FEBS Let. 455:70-74.
Tautz, 1989, Hypervariability of simple sequences as a general source for polymorphic DNA markers. Nucleic Acids Res. 17:6563-6571.
Thallman R M, G L Bennett, J W Keele and S M Kappes. 2001a. Efficient computation of genotype probabilities for loci with many alleles: I. Allelic peeling. J Anim Sci 79:26-33.
Thallman R M, G L Bennett, J W Keele and S M Kappes. 2001b. Efficient computation of genotype probabilities for loci with many alleles: II. Iterative method for large, complex pedigrees. J Anim Sci 79:34-44.
Walker et al., “Strand displacement amplification—an isothermal, in vitro DNA amplification technique,” Nucleic Acids Res. 20(7):1691-1696, 1992
Weber, J. L. and P. E. May. 1989. Abundant class of human DNA polymorphisms which can be typed using the polymerase chain reaction. Amer. J. Human Genet. 44:388-396.
White S N, K H Taylor, C A Abbey, C A Gill and J E Womack. 2003. Haplotype variation in bovine Toll-like receptor 4 and computational prediction of a positively selected ligand-binding domain. Proc Natl Acad Sci USA 100: 10364-10369.

What is claimed is:

1. A method of determining the genetic predisposition of a female head of beef cattle for increased weaning weight of the progeny of the head of beef cattle comprising genotyping the head of beef cattle to determine the genotype for DGAT1.

2. The method of claim 1, wherein genotyping comprises determining the genotype of at least one parent of said head of beef cattle.

3. The method of claim 2, comprising genotyping both parents of said head of beef cattle.

4. The method of claim 1, wherein said head of beef cattle is a Bos indicus or Bos taurus head of beef cattle.

5. The method of claim 1, wherein said head of beef cattle is an Angus beef cattle.

6. The method of claim 1, further defined as comprising genotyping a population of beef cattle for DGAT1.

7. The method of claim 6, further comprising identifying at least a first head of beef cattle from the population comprising a K232A polymorphism in DGAT1.

8. The method of claim 7, further comprising breeding the first head of beef cattle from the population comprising a K232A polymorphism in DGAT1 with a second head of beef cattle to obtain a progeny head of beef cattle with an increased weaning weight relative to a progeny of a female head of beef cattle of the same breed lacking the polymorphism.

9. The method of claim 1, wherein genotyping is carried out by assaying of genetic material from the head of beef cattle.

10. The method of claim 1, wherein genotyping is carried out by PCR.

11. The method of claim 1, wherein genotyping is carried out by nucleic acid hybridization.

12. The method of claim 9, wherein the genetic material is from a gamete.

13. The method of claim 9, wherein the genetic material is genomic DNA.

14. A method of breeding beef cattle to increase weaning weight, comprising the steps of:
(a) assaying at least one candidate female head of beef cattle to identify a first female parent head of beef cattle comprising a genetic polymorphism in DGAT1 that confers increased weaning weight in progeny of the head of beef cattle; and
(b) breeding the first parent head of beef cattle with a second parent head of beef cattle to obtain a progeny head of beef cattle with an increased weaning weight relative to a progeny of a female head of beef cattle of the same breed lacking the polymorphism.

15. The method of claim 14, wherein the second parent head of beef cattle comprises said genetic polymorphism.

16. The method of claim 14, further defined as comprising crossing said progeny head of beef cattle with a third head of beef cattle to produce a second generation progeny head of beef cattle.

17. The method of claim 14, wherein said first parent head of beef cattle is selected from a progeny head of beef cattle resulting from a previous repetition of said step (a) and said step (b) and wherein said second parent head of beef cattle is from a selected cattle breed into which one wishes to increase the occurrence of said polymorphism.

18. The method of claim 17, further defined as comprising repeating step (a) and step (b) from about 2 to about 10 times.

19. A method of breeding beef cattle comprising:
(a) assaying a population of beef cattle for the presence of a K232A polymorphism in DGAT1 associated with increased weaning weight in progeny of female beef cattle comprising the polymorphism;
(b) selecting members of the population comprising the K232A polymorphism; and
(c) breeding the selected members of the population to produce progeny beef cattle.

20. The method of claim 19 further comprising:
(a) assaying the progeny beef cattle for the presence of a K232A polymorphism in DGAT1 associated with increased weaning weight in progeny of female beef cattle comprising the polymorphism;
(b) selecting progeny beef cattle comprising the K232A polymorphism; and
(c) breeding the selected progeny beef to produce progeny beef cattle of a subsequent generation.
